(19) United States
(12) Patent Application Publication
(10) Pub. No.: US 2019/0060508 A1
(43) Pub. Date: Feb. 28, 2019

(54) WOUND DRESSING AND METHOD FOR REMOVING THE SAME

(71) Applicant: HON HAI PRECISION INDUSTRY CO., LTD., New Taipei (TW)

(72) Inventor: Hsiu-Wen Chien, New Taipei (TW)

(21) Appl. No.: 15/833,081

(22) Filed: Dec. 6, 2017

(30) Foreign Application Priority Data
Aug. 31, 2017 (TW) 106129787

(51) Int. Cl. A61L 15/26; A61F 13/00; A61N 5/06; A61L 15/44; A61L 15/42

(52) U.S. Cl. CPC A61L 15/26; A61F 13/00017; A61N 5/062; A61F 13/00042; A61L 2300/404; A61L 15/44; A61L 15/425; A61L 15/42; A61N 2005/0663; A61F 13/00063

(57) ABSTRACT

A wound dressing which is removable with little or no adhesion to wounded tissue includes a wound contact layer. The wound contact layer comprises molecules which can be rapidly degraded by exposure to certain light, and non-sticking molecules. The photodegradable molecules includes at least one o-nitrobenzyl group.

### FIELD

The subject matter generally relates to medical technology, a wound dressing, and a method for removing the wound dressing from a wound.

### BACKGROUND

A wound dressing may be necessary for attending to injuries. Typical wound dressings consist of three layers, a wound contact layer, an absorbent layer, and a hydrophobic layer. Wound contact layer is placed on an open wound to protect tissue. The wound bed suppurates, the protein, blood, and others substances, which are dried out and become an adhesive. Thus, achieving 100% non-stick coating is difficult, and the injured always feels pain when the dressing is being removed. Improvement in the art is preferred.

### BRIEF DESCRIPTION OF THE DRAWINGS

Implementations of the present disclosure will now be described, by way of example only, with reference to the attached figures.

FIG. 1 is a perspective view of an exemplary embodiment of a wound dressing according to the present disclosure.

FIG. 2 is a flowchart of a method for removing the wound dressing of FIG. 1.

### DETAILED DESCRIPTION OF EMBODIMENTS

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to better illustrate details and features of the present disclosure.

The disclosure is illustrated by way of example and not by way of limitation in the figures of the accompanying drawings. It should be noted that references to “an” or “one” embodiment in this disclosure are not necessarily to the same embodiment, and such references mean “at least one.”

The term “comprising” when utilized, means “including, but not necessarily limited to”; it specifically indicates open-ended inclusion or membership in the so-described combination, group, series, and the like.

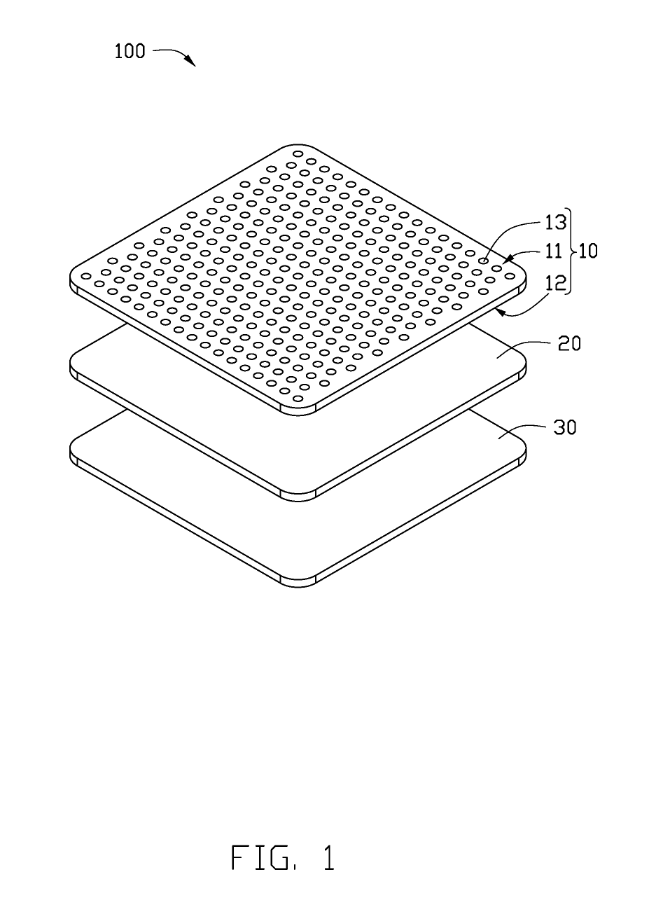

FIG. 1 illustrates an exemplary embodiment of a wound dressing 100. The wound dressing 100 includes a wound contact layer 10, an absorbent layer 20, and a hydrophobic layer 30. The absorbent layer 20 is formed between the wound contact layer 10 and the hydrophobic layer 30.

The wound contact layer 10 includes a wound contact surface 11 and a non-wound contact surface 12. The non-wound contact surface 12 faces away from the wound contact surface 11. The absorbent layer 20 is formed on the non-wound contact surface 12. The hydrophobic layer 30 is formed on the absorbent layer 20 and faces away from the non-wound contact surface 12.

A number of flow-guiding holes 13 are formed in the wound contact layer 10. The flow-guiding holes 13 run through the wound contact layer 10.

In at least one exemplary embodiment, the flow-guiding holes 13 formed in the wound contact layer 10 are array in arrangement.

The flow-guiding holes 13 are used to guide the flow of the suppurations produced by wound bed to the absorbent layer 20.

The wound contact layer 10 is made of photodegradable molecules and non-sticking molecules.

The photodegradable molecules facilitate removal of the wound dressing 100.

The photodegradable molecules include at least one o-nitrobenzyl group. The o-nitrobenzyl group can be degraded by exposure to light with a wavelength of 405 nanometers.

The photodegradation reaction equation of the o-nitrobenzyl group in the wound contact layer 10 is:

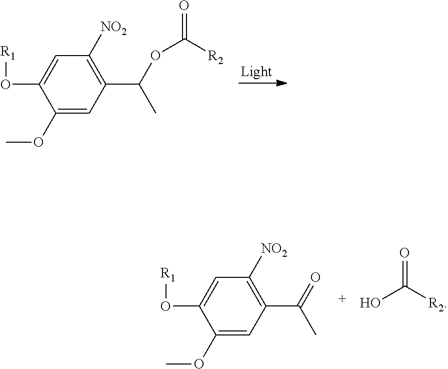

The non-sticking molecules prevent the wound dressing 100 to stick in the wound bed.

The non-sticking molecules can be polyethylene glycol (PEG), or polycarbixybetaine methacylate (PCBMA), or polysulfobetaine methacrylate (PSBMA), or poly(methacryloyloxyethyl phosphorylcholine) (PMPC), or polyacrylamide (PAAM), or poly-2-methyl-2-oxazoline (PMOXA), or others.

The molecular structural formula of the PEG is:

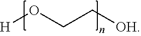

The molecular structural formula of the PCBMA is:

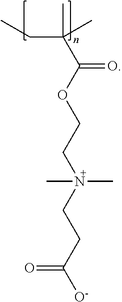

The molecular structural formula of the PSBMA is:

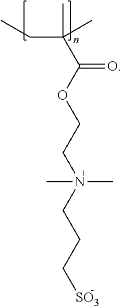

The molecular structural formula of the PMPC is:

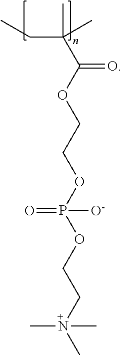

The molecular structural formula of the PAAM is:

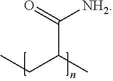

The molecular structural formula of the PMOXA is:

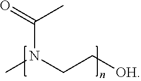

In at least one exemplary embodiment, the non-sticking molecules are PEG molecules. The photodegradable molecules include two o-nitrobenzyl groups. In the wound contact layer 10, the two o-nitrobenzyl groups are bonded to one PEG molecule. In at least one exemplary embodiment, the molecular structural formula of the wound contact layer 10 is:

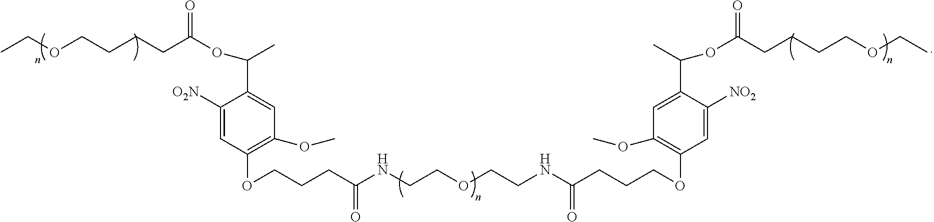

Since the PEG molecules have non-foul characteristic, the photodegradable molecules can allow for the wound dressing 100 to not stick in the wound bed.

The wound contact layer 10 further includes antibacterial medicine, wound healing medicine, and the like to promote rapid healing of the wound.

The absorbent layer 20 can be used to absorb liquid or semi-liquid matter, to reduce serious risk of pollution or infection, and to promote rapid healing of the wound.

Material of the absorbent layer 20 can be hydrophilic polymer foam, absorbent web, non-woven fabric composing cotton fibers, viscose fibers, and polyester fibers, polymer matrix containing at least one hydrocolloid, lyophilized bubbles, and a combination thereof.

In at least one exemplary embodiment, the material of the absorbent layer 20 is an absorbent web.

The hydrophobic layer 30 prevents ingress of water, moisture, impurities, dust, bacteria and other pollutants into the wound, so as not to affect the healing of the wound.

The hydrophobic layer 30 can use any material common in modern trauma therapy that is used as a hydrophobic layer.

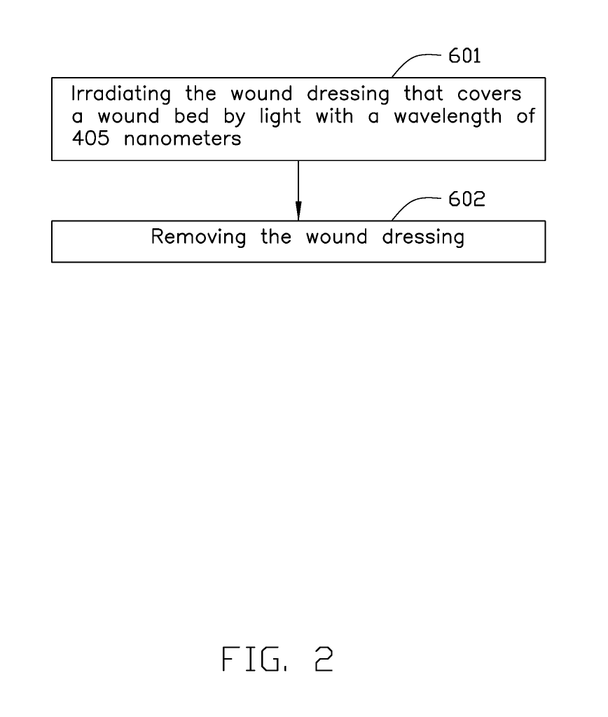

FIG. 2 illustrates a flowchart of a method for removing the wound dressing 100. The method is provided by way of example, as there are a variety of ways to carry out the method. Each block shown in FIG. 2 represents one or more processes, methods, or subroutines, carried out in the exemplary method. Furthermore, the illustrated order of blocks is by example only and the order of the blocks can change. Additional blocks may be added or fewer blocks may be utilized, without departing from this disclosure. The exemplary method can begin at block 601.

At block 601, also illustrated by FIG. 2, the wound dressing 100 covering the wound bed is irradiated by light with a wavelength of 405 nanometers.

At block 602, also illustrated by FIG. 2, the wound dressing 100 is removed.

With the above configuration, the photodegradable molecules in the wound contact layer 10 degrade rapidly on exposure to light with a wavelength of 405 nanometers, so the wound contact layer 10 can be simply and rapidly removed. The non-sticking molecules in the wound contact layer 10 prevent the wound dressing 100 to stick in the wound bed. The wound dressing 100 can be removed with little or no pain.

The embodiments shown and described above are only examples. Many details are often found in the art such as the other features of the wound dressing having the same. Therefore, many such details are neither shown nor described. Even though numerous characteristics and advantages of the present disclosure have been positioned forth in the foregoing description, together with details of the structure and function of the present disclosure, the disclosure is illustrative only, and changes can be made in the detail, including in matters of shape, size, and arrangement of the parts within the principles of the present disclosure, up to and including the full extent established by the broad general meaning of the terms used in the claims. It will therefore be appreciated that the embodiments described above can be modified within the scope of the claims.

What is claimed is:

1. A wound dressing comprising:
a wound contact layer, wherein the wound contact layer comprises:
photodegradable molecules and non-sticking molecules, wherein the photodegradable molecules comprises at least one o-nitrobenzyl group.

2. The wound dressing of claim 1, wherein the o-nitrobenzyl group is photodegraded by exposing to light with a wavelength of about 405 nanometers.

3. The wound dressing of claim 1, wherein the wound contact layer has a molecular structural formula of:

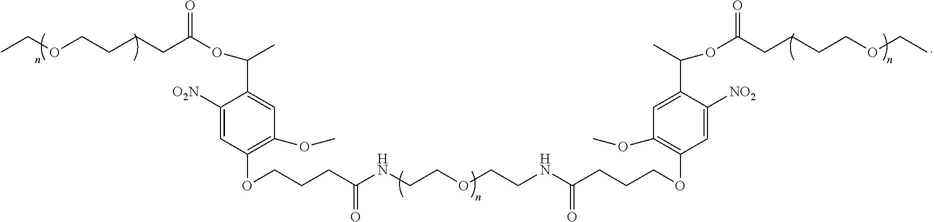

4. The wound dressing of claim 1, wherein the non-sticking molecules is a material selected from the group consisting of polyethylene glycol 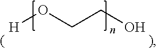
polycarbixybetaine methacylate 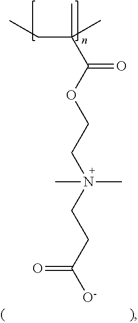
polysulfobetaine methacrylate 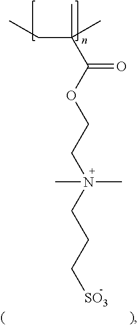
poly(methacryloyloxyethyl phosphorylcholine) 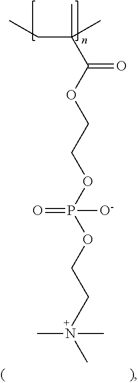
polyacrylamide 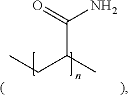
poly-2-methyl-2-oxazoline 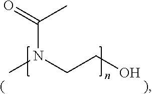
and a combination thereof.

5. The wound dressing of claim 1, wherein the wound contact layer comprises a wound contact surface and a non-wound contact surface, and the non-wound contact surface faces away from the wound contact surface.

6. The wound dressing of claim 5, wherein the wound contact layer further comprises antibacterial medicine, wound healing medicine, or a combination thereof on the wound contact surface.

7. The wound dressing of claim 5, wherein the wound dressing further comprises an absorbent layer, the absorbent layer is formed on the non-wound contact surface.

8. The wound dressing of claim 7, wherein the absorbent layer has a material selected from the group consisting of hydrophilic polymer foam, absorbent web, non-woven fabric comprising cotton fibers, viscose fibers and polyester fibers, polymer matrix containing at least one hydrocolloid, lyophilized bubbles, and a combination thereof.

9. The wound dressing of claim 7, wherein the wound dressing further comprises a hydrophobic layer, the hydrophobic layer is formed on the absorbent layer and faces away from the non-wound contact surface.

10. The wound dressing of claim 7, wherein a plurality of flow-guiding holes are formed in the wound contact layer, the flow-guiding holes run through the wound contact layer.

11. A method for removing a wound dressing, comprising:
irradiating, by light, a wound bed covered by the wound dressing; the wound dressing comprising a wound contact layer, wherein the wound contact layer comprises photodegradable molecules and non-sticking molecules, wherein the photodegradable molecules comprising at least one o-nitrobenzyl group; and
removing the wound dressing.

12. The method of claim 11, wherein a wavelength of the light is 405 nanometers.

13. The method of claim 11, wherein the wound contact layer has a molecular structural formula of:

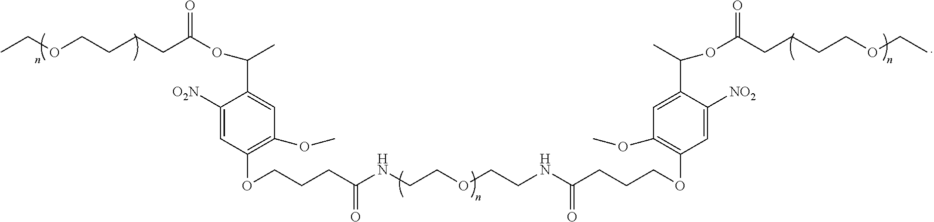

14. The method of claim 11, wherein the non-sticking molecules is a material selected from the group consisting of polyethylene glycol 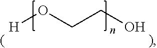
or polycarbixybetaine methacylate 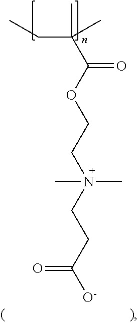
or polysulfobetaine methacrylate 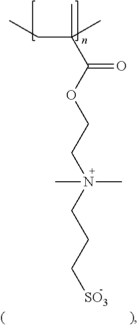
or poly(methacryloyloxyethyl phosphorylcholine) 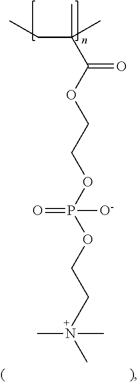
or polyacrylamide 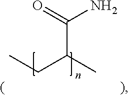
or poly-2-methyl-2-oxazoline 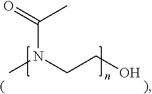
or others.

15. The method of claim 11, wherein the wound contact layer comprises a wound contact surface and a non-wound contact surface, and the non-wound contact surface faces away from the wound contact surface.

16. The method of claim 15, wherein the wound contact layer further comprises antibacterial medicine, wound healing medicine, or a combination thereof on the wound contact surface.

17. The method of claim 15, wherein the wound dressing further comprises an absorbent layer, the absorbent layer is formed on the non-wound contact surface.

18. The method of claim 17, wherein the absorbent layer has a material selected from the group consisting of hydrophilic polymer foam, absorbent web, non-woven fabric composing cotton fibers, viscose fibers and polyester fibers, polymer matrix containing at least one hydrocolloid, and a combination thereof.

19. The method of claim 17, wherein the wound dressing further comprises a hydrophobic layer, the hydrophobic layer is formed on the absorbent layer and faces away from the non-wound contact surface.

20. The method of claim 17, wherein a plurality of flow-guiding holes is formed in the wound contact layer, the flow-guiding holes run through the wound contact layer.
